(19) United States
(12) Patent Application Publication
(10) Pub. No.: US 2026/0284401 A1
(43) Pub. Date: Sep. 24, 2026

(54) SEARCH AND OPTIMIZATION ALGORITHMS FOR EXPLORING NOVEL STIMULATION PATTERNS

(71) Applicant: Boston Scientific Neuromodulation Corporation, Valencia, CA (US)

(72) Inventors: Jianwen Gu, Richmond, TX (US); Hemant S Bokil, Cambridge, MA (US); Mahsa Malekmohammadi, Sherman Oaks, CA (US)

(73) Assignee: Boston Scientific Neuromodulation Corporation, Valencia, CA (US)

(21) Appl. No.: 19/570,782

(22) Filed: Mar. 18, 2026

Related U.S. Application Data
(60) Provisional application No. 63/774,562, filed on Mar. 19, 2025.

(51) Int. Cl. A61N 1/36; A61N 1/05

(52) U.S. Cl. CPC A61N 1/36135; A61N 1/0534; A61N 1/36178

(57) ABSTRACT

Methods and systems configured for identifying effective therapy regimens and optimizing output settings. One or more test stimulations are issued to a patient having a neuromodulation system, and a relationship between at least one test parameter and at least on therapy effect is identified. Optimization uses this relationship to configure therapy parameters to cause a desired therapy effect.

### CROSS REFERENCE TO RELATED APPLICATIONS

This application claims the benefit of U.S. Provisional Patent Application Ser. No. 63/774,562 filed on Mar. 19, 2025, the disclosure of which is incorporated herein by reference.

### BACKGROUND

Neuromodulation systems issue electrical pulses at targeted anatomy (the brain, spinal cord, vagus nerve and/or various peripheral nerves) to affect neural function. Earlier versions of neuromodulation systems could issue tonic stimulation at a fixed pulse repetition rate using a selected pair of electrodes for a predetermined period of time, at a fixed amplitude. Neuromodulation systems have become more sophisticated over time, providing options for precise targeting using concepts such as current or voltage steering, with wider ranges of available frequencies and more complex patterns of stimulation. Burst stimulation, for example, provides groups or “bursts” of pulses using an intraburst period, separated by interburst periods. As more complex patterns can be used, more variables are present, making programming of these systems more complicated and time consuming. New and enhanced approaches to identifying effective therapy regimens and optimizing output settings are desired.

### OVERVIEW

The present inventors have recognized, among other things, that a problem to be solved is the need for new and/or alternative approaches to identifying effective therapy regimens and optimizing output settings.

A first illustrative and non-limiting example takes the form of a method of treating a patient with an implantable neuromodulation system having a pulse generator and at least one lead having a plurality of electrodes thereon, the method comprising: delivering a first programmed therapy to the patient with first parameters using the pulse generator and at least one lead; observing a first response to the delivered first programmed therapy; delivering a second programmed therapy to the patient with second parameters using the pulse generator and at least one lead, the second parameters having at least one changed parameter relative to the first parameters; observing a second response to the delivered second programmed therapy; identifying a relationship between the changed parameter and a therapy response from the first programmed therapy, the first response, the second programmed therapy, and the second response; calculating an optimized therapy using the identified relationship; and delivering the optimized therapy to the patient.

Additionally or alternatively, the therapy response is a persistence of a therapy effect determined as a length of time from when a therapy output stops to when the therapy effect stops. Additionally or alternatively, the first programmed therapy is a burst therapy having an interburst period, an intraburst period, and a number of stimuli in each burst, and the changed parameter is the interburst period. Additionally or alternatively, the first programmed therapy is a burst therapy having an interburst period, an intraburst period, and a number of stimuli in each burst, and the changed parameter is the intraburst period. Additionally or alternatively, the first programmed therapy is a burst therapy having an interburst period, an intraburst period, and a number of stimuli in each burst, and the changed parameter is the number of stimuli in each burst. Additionally or alternatively, the first programmed therapy is a burst therapy comprising a series of bursts including a first burst and a last burst, and the time when the therapy output stops is at the end of the last burst.

Additionally or alternatively, the therapy response is a delay to onset of a therapy effect determined as a length of time when a therapy output starts to when the therapy effect starts. Additionally or alternatively, the therapy response is determined from a measure of patient movement determined using one or more of a wearable device, or a motion sensor of the implantable neuromodulation system.

Additionally or alternatively, the implantable neuromodulation system is a deep brain stimulation system having the at least one lead implanted in the patient's brain, and the therapy response is determined from a measurable electrical activity in the brain of the patient measured either with the implantable neuromodulation system or by a separate device.

Additionally or alternatively, the first programmed therapy is output with a first spatial pattern of stimulation, and the second programmed therapy is output with a second spatial pattern of stimulation, the changed parameter is the spatial pattern, and the relationship characterizes a therapy effect as a function of spatial pattern of stimulation. Additionally or alternatively, the first spatial pattern has a first spread of stimulation outputs, the second spatial pattern has a second spread of stimulation outputs, and the relationship characterizes the therapy effect as a function of spread of stimulation outputs. Additionally or alternatively, the first spatial pattern has a first center of stimulation outputs, and the second spatial pattern as a second center of stimulation outputs, and the relationship characterizes the therapy effect as a function of the center of stimulation outputs.

Additionally or alternatively, the relationship characterizes a persistence of the therapy effect as a function of spatial and temporal parameters of the stimulation outputs.

Another illustrative and non-limiting example takes the form of a method of treating a patient with an implantable neuromodulation system having a pulse generator and at least one lead having a plurality of electrodes thereon, the method comprising: testing a first programmed therapy by: delivering the first programmed therapy to the patient; observing a first change in a chosen symptom of the patient; ceasing to deliver the first programmed therapy; observing a second change in the chosen symptom of the patient; and determining a first duration related to the first programmed therapy and the chosen symptom; testing a second programmed therapy by: delivering the second programmed therapy to the patient; observing a third change in the chosen symptom of the patient; ceasing to deliver the second programmed therapy; observing a fourth change in the chosen symptom of the patient; and determining a second duration related to the first programmed therapy and the chosen symptom; identifying a relationship between a therapy parameter and a therapy result from the first programmed therapy, the first duration, the second programmed therapy, and the second duration; calculating an optimized therapy using the identified relationship; and delivering the optimized therapy to the patient.

Additionally or alternatively, the pulse generator is configurable to deliver electrical stimuli using a plurality of spatial patterns, the first programmed therapy is output with a first spatial pattern, and the second programmed therapy is output with a second spatial pattern different from the first spatial pattern, and the relationship characterizes a therapy effect as a function of spatial pattern of stimulation.

Additionally or alternatively, the first spatial pattern has one of: a first central point of stimulation which is different from a second central point of stimulation of the second spatial pattern; or a first spread of electrical outputs in the plurality of electrodes which is different form a second spread of electrical outputs in the plurality of electrodes.

Additionally or alternatively, each of the first programmed therapy and the second programmed therapy are burst therapies each having an interburst period, an intraburst period, and a number of stimuli in each burst, and wherein the first programmed therapy uses a first interburst period and the second programmed therapy uses a second interburst period different from the first interburst period. Additionally or alternatively, each of the first programmed therapy and the second programmed therapy are burst therapies each having an interburst period, an intraburst period, and a number of stimuli in each burst, and wherein the first programmed therapy uses a first intraburst period and the second programmed therapy uses a second intraburst period different from the first intraburst period. Additionally or alternatively, each of the first programmed therapy and the second programmed therapy are burst therapies each having an interburst period, an intraburst period, and a number of stimuli in each burst, and wherein the first programmed therapy uses a first number of stimuli in each burst and the second programmed therapy uses a second number of stimuli in each burst different from the first number of stimuli in each burst.

Another illustrative and non-limiting example takes the form of an implantable neuromodulation system comprising: an implantable pulse generator; at least one implantable lead having a plurality of electrodes thereon; and an external programmer adapted to communicate with and provide therapy instructions to the implantable pulse generator, wherein the system is configured to: deliver a first programmed therapy to the patient with first parameters using the pulse generator and at least one lead; observe a first response to the delivered first programmed therapy; deliver a second programmed therapy to the patient with second parameters using the pulse generator and at least one lead, the second parameters having at least one changed parameter relative to the first parameters; observe a second response to the delivered second programmed therapy; identify a relationship between the changed parameter and a therapy response from the first programmed therapy, the first response, the second programmed therapy, and the second response; calculate an optimized therapy using the identified relationship; and deliver the optimized therapy to the patient.

Additionally or alternatively, the therapy response is a persistence of a therapy effect determined as a length of time from when a therapy output stops to when the therapy effect stops.

Additionally or alternatively, the first programmed therapy is a burst therapy having an interburst period, an intraburst period, and a number of stimuli in each burst, and the changed parameter is the interburst period. Additionally or alternatively, the first programmed therapy is a burst therapy having an interburst period, an intraburst period, and a number of stimuli in each burst, and the changed parameter is the intraburst period. Additionally or alternatively, the first programmed therapy is a burst therapy having an interburst period, an intraburst period, and a number of stimuli in each burst, and the changed parameter is the number of stimuli in each burst. Additionally or alternatively, the first programmed therapy is a burst therapy comprising a series of bursts including a first burst and a last burst, and the time when the therapy output stops is at the end of the last burst.

Additionally or alternatively, the therapy response is a delay to onset of a therapy effect determined as a length of time when a therapy output starts to when the therapy effect starts.

Additionally or alternatively, the pulse generator comprises a motion sensor configured to measure patient movement, wherein the therapy response is determined from a measure of patient movement determined using the motion sensor, the external programmer configured to communicate with the pulse generator to obtain the measure of patient movement, or, the system further comprises a wearable device configured to measure patient movement, wherein the therapy response is determined from a measure of patient movement determined using the wearable device, the external programmer configured to communicate with the wearable device to obtain the measure of patient movement.

Additionally or alternatively, the implantable neuromodulation system is a deep brain stimulation system having the at least one lead implanted in the patient's brain, and the therapy response is determined from a measurable electrical activity in the brain of the patient measured either with the implantable neuromodulation system or by a separate device.

Additionally or alternatively, the first programmed therapy is output with a first spatial pattern of stimulation, and the second programmed therapy is output with a second spatial pattern of stimulation, and the relationship characterizes a therapy effect as a function of spatial pattern of stimulation. Additionally or alternatively, the first spatial pattern has a first spread of stimulation outputs, the second spatial pattern has a second spread of stimulation outputs, and the relationship characterizes the therapy effect as a function of spread of stimulation outputs. Additionally or alternatively, the first spatial pattern has a first center of stimulation outputs, and the second spatial pattern as a second center of stimulation outputs, and the relationship characterizes the therapy effect as a function of the center of stimulation outputs.

Additionally or alternatively, the relationship characterizes a persistence of the therapy effect as a function of spatial and temporal parameters of the stimulation outputs.

Additionally or alternatively, the pulse generator delivers the first programmed therapy, the second programmed therapy, and the optimized therapy using the lead; the system is configured observe the first and second responses by one of: requesting a patient input; or measuring a signal using a wearable device, the pulse generator, or an ingestible or implantable device separate from the pulse generator; the external programmer identifies the relationship and determines a quantification thereof; the external programmer calculates the optimized therapy using the quantification of the relationship.

Additionally or alternatively, the external programmer identifies the relationship and determines a quantification thereof and applies a cost function to calculate the optimized therapy using the quantification of the relationship.

This overview is intended to introduce the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative neuromodulation system;

FIG. 2 shows an illustrative lead with segmented electrodes;

FIGS. 3-4 graphically illustrate tonic and burst stimulation, respectively; and

FIGS. 5-7 are block diagrams for illustrative methods.

### DETAILED DESCRIPTION

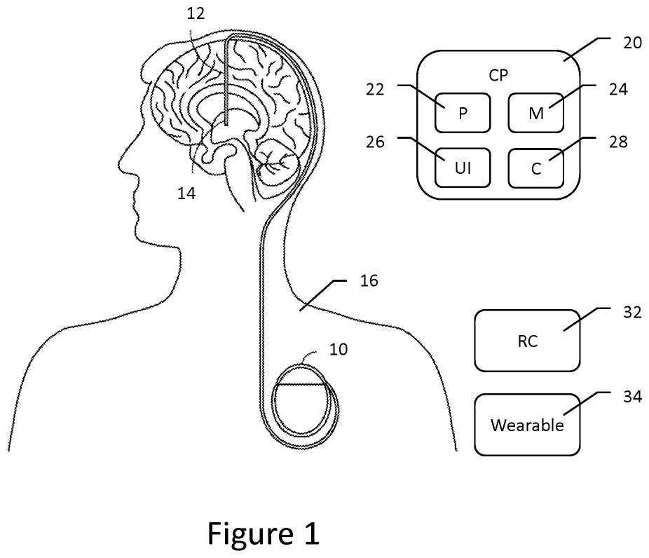

FIG. 1 shows an illustrative neuromodulation system implanted in a patient. The present concepts may be used in a range of neuromodulation products, including deep brain stimulation (DBS), spinal cord stimulation (SCS), vagus nerve stimulation (VNS), sacral nerve stimulation (SNS), and/or others. In the example of FIG. 1, a DBS system is shown implanted in a patient.

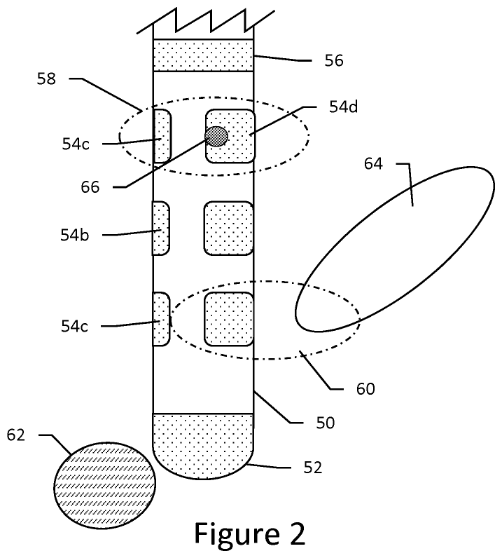

The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the hippocampus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, seizure disorders including epilepsy, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as determining seizure frequency, or monitoring gait or tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 20 can be used by a physician, or at the direction of a physician, to obtain data from and provide instructions the pulse generator 10 via suitable communications protocols such as Bluetooth or MedRadio or other wireless communications standards, and/or via other modalities such as inductive telemetry. To these ends, the CP 20 is shown including a processor 22, a memory 24, a user interface 26, and communication circuitry 28. The CP may, for example and without limitation, be a tablet or laptop computer, using the standard processor 22 and user interface 26 for such commercial, off-the shelf systems, with a communication circuit 28 which can be a standard communications circuit (such as Bluetooth) or may be special purpose and/or custom, such as using MedRadio band or other communications protocol and frequency. The memory 24 may store instructions for operating the system in the methods described herein in any suitable transitory and/or non-transitory media.

The skilled person will recognize many different hardware implementations are available for a controller and/or processor. A processor 22 may take many forms, including, for example, a microcontroller or microprocessor, coupled to a memory storing readable instructions for performing methods as described herein, as well as providing configuration of the processor for the various examples that follow. The word “processor” should not be taken as limiting to only microprocessors, for example, and may encompass microcontrollers and/or state-machine architectures. The processor may include one more application-specific integrated circuits (ASIC) to provide additional or specialized functionality, such as, without limitation a signal processing ASIC that can filter received signals from one or more sensors via digital filtering techniques. Logic circuitry, state machines, and discrete or integrated circuit components may be included as well.

A patient remote control (RC) 32 can be used by the patient to perform various actions relative to the pulse generator 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 20 to control and/or obtain data from the pulse generator 10. The patient RC 32 may also be programmable on its own or may communicate or be linked with the CP 20. The patient RC may be software defined, that is, taking the form of an executable set of instructions stored on non-transitory media, such an application for execution on a smartphone. For example, the patient RC may be any of a special purpose device, or a general purpose off-the-shelf device such as a tablet computer or smartphone operating an application (which may lock the tablet computer or smartphone for use only as the patient RC, or which may be operable along with consumer apps and functionality).

A charger (not shown) may be provided to the patient to allow the patient to recharge the pulse generator 10, if the pulse generator 10 is rechargeable. Some pulse generators 10 (referred to as primary cell devices) are not rechargeable, and so the charger may be omitted. The charger can operate, for example, by generating a varying magnetic field to activate an inductor associated with the pulse generator 10 to provide power to recharge the pulse generator battery, using known methods and circuitry.

Some systems may include an external test stimulator (ETS—not shown), which may be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS, and the ETS can be programmed using the CP 20 with various therapy programs and stimulation parameters. Once therapy suitability for the patient is established to the satisfaction of the patient 16 and/or physician, the permanent IPG 10 is implanted and the lead 12 is connected thereto, with the ETS then removed from use. Additional components, such as a remote monitor or bedside monitor may also be included.

The pulse generator 10 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for SCS, VNS, SNS, and/or DBS. Additional background and/or examples of the pulse generator 10, CP 20, RC 32, charger, and ETS can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The system may also be configured for using and/or communicating with wearable device 34. A wearable device 34 may be used to track or otherwise observe patient activity. Step counters, fitness watches, heart rate monitors, and other commercially available systems can be used to track and observe patient activity, which may in turn be used to determine how and when a patient responds to delivered therapy. Some examples may use less widely available wearables 34, such as those available as medical devices and/or by prescription to track or observe patient movement, activity levels, and other responses to therapy. Wearable devices 34 may track, for example and without limitation, gross or fine patient movement, tremor, steps taken, heart rate, other cardiac function, galvanic skin response, any other electrical signal, including brain waves and the like. Skin or other temperatures as well as glucose or other blood constituent levels may be tracked. Some examples may use secondary implantable systems to observe patient response to a therapy. This may include, for example, in a DBS or other neuromodulation system, monitoring for neural response to therapy (such as action potentials, including compound action potentials), monitoring hormonal or other excretions and levels, monitoring for organ activities including intentional and non-intentional movement (intestinal tract activity, breathing including negatively connotated breathing activity such as rales and snoring), muscle tone, rigidity, spastic muscle activity, seizure activity, etc. Indeed, block 34 may represent wearable, ingestible, and/or implantable devices and/or sensors of the system itself that can track any patient response to the therapy delivered by the system Oct. 12, 2020.

FIG. 2 illustrates details of a directional DBS lead 50. The distal end is shown, and a plurality of electrodes are shown as well. The device is shown with a ring electrode 56 and a tip electrode 52, with segmented electrodes 54a, 54b, and 54c (collectively, segmented electrodes 54) therebetween in which may be characterized as a 1-3-3-1 configuration. Each electrode 52, 54, 56 may be separately addressable in the system, such as by using a pulse generator having multiple independent current control (MICC) or multiple voltage sources. The configuration shown is merely illustrative, and other designs, including 16-contact directional leads, can be used.

MICC is a stimulus control system that uses multiple independent current sources to provide a plurality of independently generated, separately controllable, output currents that may each have an independent quantity of current. The use of MICC can allow spatially selective fields to be generated during therapy outputs. The term “fractionalization” may refer to how the total current issued by the pulse generator via the electrodes is divided up amongst the electrodes 52, 54, 56 on the lead. It should be noted that the pulse generator canister may serve as an indifferent electrode or as a return electrode for therapy outputs. Alternatively, one of the lead electrodes (such as a ring electrode 56) may instead be used as a return electrode. Thus, for example, the electrodes 52, 54, 56 on the lead may serve as cathodes while pulse generator canister serves as an anode during one phase of stimulation pulse delivery. In another example, some of the lead electrodes 52, 54, 56 serve as cathodes, while other lead electrodes 52, 54, 56 serve as anodes during one phase of stimulation pulse delivery.

Examples of electrical leads with segmented or directional lead structures are shown, for example and without limitation, in US PG Pat. Pubs. 20100268298, 20150021817, 20150066120, and 20150151113, and U.S. Pat. No. 8,321,025, the disclosures of which are incorporated herein by reference.

A directional lead as shown in FIG. 2 may be used to generate a stimulation field as illustrated at 58 and/or 60 in FIG. 2. The outer boundary of each field may be understood as representing an equipotential or equal field boundary equal to the estimated activation threshold of the neural tissue. Inside the boundary, the electrical field is higher than an activation threshold, and outside of the boundary the electrical field is below the threshold, for purposes of illustration. The determination or estimate of the neural activation threshold may represent or approximate a voltage/field strength at which neural cells will activate, on average, across a population of people and neural cellular structures. Activation thresholds may be determined on a population basis, such as by relating to a voltage/field at which a 50% likelihood of activation of 50% of the cell population is determined; other boundaries/thresholds can be used. The shape of the field can be adjusted, as described variously in the MICC-related references incorporated by reference above, by modifying the fractionalization of current issued via the electrodes. An electrical field as shown at 58 may be (roughly) generated by using electrode 54d as a cathode, and surrounding electrodes at 54c, or level with 54b and/or ring 56 as anodes, for example. The actual characteristics of fractionalization may be more sophisticated than this simple example.

The central point of stimulation is a variable or parameter of stimulation which can be used to describe how the stimulation field is directed. For example, with the stimulation field shown at 58, the mathematical center of stimulation outputs can be determined as shown at 66 as a mean or average of the output cathodic (or alternatively, anodic) currents. Spread refers to how current surrounding the central point of stimulation 66 is distributed. A “narrow” spread would have a smaller outer boundary for the field shown at 58; this means that the output currents are more focused in the vicinity of the central point of stimulation. If currents are more broadly spread across more electrodes, this would be a wider “spread” of the stimulation outputs. If the sum of all currents is the same, a narrow spread would have a smaller area within the boundary shown at 58, with higher electrical fields within the boundary relative to the activation threshold. A wider spread would have a greater area within the boundary 58, but the electric fields would generally be lower than with a narrower spread. Moving the central point of stimulation around will cause the stimulation field to move in space, while changing spread will expand or contract the size of the boundary 58 around the central point of stimulation 66.

A related concept to the fields shown at in FIG. 2 is that of stimulation field modeling (SFM). In SFM, the tissue is modeled, for example, using finite element models in which the lead body is treated as an insulator, surrounded by a thin encapsulation sheath, and further surrounded by neural tissue. The neural tissue may be modeled as isotropic and homogenous, though more sophisticated modelling can also be used if desired. A set of model volumes are defined around the lead, breaking up the space into small blocks, each of which can be analyzed within the model. The outer boundaries of the SFM can be determined using a population-based activation threshold. The result can be that at a given stimulation current, an SFM can be generated as a three-dimensional surface surrounding a portion of the lead and encompassing a volume of neural tissue. Fields 58 and 60 may be, for example, understood as a two-dimensional representation of a slice of the SFM.

The positioning of the lead (and related SFM) relative to target and/or non-target (side-effect) neural structures may be determined for a given system using imaging modalities such as X-ray, CT scan, PET scan, MRI, f-MRI, etc., to identify lead position in the patient. Imaging system data for the patient may be used to estimate locations of neural structures in the patient, relative to the identified lead position. The imaging data may be merged or overlapped with general anatomical knowledge, such as from a brain atlas, which can help identify the particular structures that can be identified from imaging data. The use of SFM in association with a directional lead can allow therapy planning to precisely stimulate targeted tissue structures while avoiding excess stimulation of non-target tissue and limiting stimulation of structures associated with adverse side effects.

The use of a current controlled system provides a degree of control over the resulting fields that are generated in neural tissue. Voltage controlled systems may also provide steering and spatially selective electrical fields. For example, a plurality of voltage sources can be directed to the electrodes of the system at different voltage potentials. The result would be an output electrical field having spatially selective characteristics. A challenge, however is that with voltage-controlled outputs, the voltage differences between adjacent electrodes can cause current to flow between those two electrodes, thus a “cathodic” electrode may not act as a cathode relative to an adjacent cathodic electrode if the two voltages are different, unlike with a current controlled system. While these differences exist, the present methods may be used in each of voltage controlled and current controlled systems.

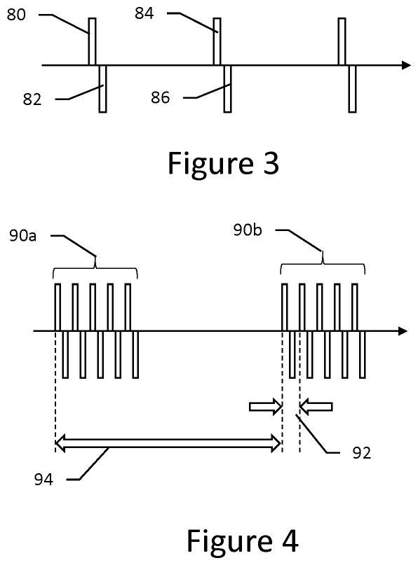

FIGS. 3-4 graphically illustrate tonic and burst stimulation, respectively. FIG. 3 shows a therapy pattern which can be referred to as tonic stimulation. A series of pulse pairs are delivered with a positive first phase 80 and a negative second phase 82. For an implanted system, the charge through an electrode is desirably zero over time, so that first phase 80 and second phase 82 may be configured with balanced charge, so that residual charge is not left at the electrode-tissue interface. An imbalanced charge can encourage degradation of the electrode and/or, if allowed to build over time, can lead to undesired stimulation effects such as muscle stimulation. For these reasons, the output is often biphasic; the second phase 82 is shown to represent an active recharge at the electrode; passive recharge is also known, in which the electrodes can be shorted to ground, for example, between positive pulse deliveries. However, monophasic pulses can be issued, if desired such as by issuing a set of monophasic pulses and grounding the outputs after the set, or by issuing a set of monophasic pulses and then reversing polarity for a subsequent set of monophasic pulses, for example and without limitation.

Each pulse has an amplitude (height, as represented), and may be a current-controlled or voltage-controlled output, as is known in the art. The pulse repetition rate is usually understood as the time from the start of the first pulse 80 to the start of the first pulse 84 of the next pulse pair. The pulse repetition rate can be reported as a frequency, for example, in Hertz. Implantable systems may issue pulses with pulse repetition rates in the range of about 0.5 to 10,000 Hz, with higher frequencies possible if desired.

FIG. 4 shows what is referred to as burst stimulation. A first group or burst of pulses is issued at 90a, as a series of N biphasic pulses (monophasic can be used if desired). Here, N=5. A second burst is shown at 90b. The period between pulses within each burst is referred to as the intraburst period, as indicated at 92. The interburst period 94 is the time from the start of one burst 90a to the start of the next burst 90b. Burst parameters can vary, including charges to intraburst period 92, interburst period 94, and the number of pulses in each burst, N.

Amplitude can vary from one pulse to the next. A common variation in amplitude is that of ramping at stimulation onset from a lower amplitude to a higher amplitude. Rampling may be used with each burst, if desired. It is also possible to vary pulse width and/or shape of the pulses. For example, while square waves are often used in illustrations, other shapes including (without limitation) sinusoidal or triangular pulse shapes can be used if desired.

Taken together, the discussion of FIGS. 2-4 shows that there are many different parameters that can be varied, including the following:
the center point of stimulation;
the spread of stimulation;
the amplitude of stimulation;
the pulse width of each output;
use of active recharge or passive recharge for each biphasic pulse;
use of monophasic or biphasic pulses;
amplitude of each pulse;
pulse width of each pulse;
pulse shape;
frequency or pulse repetition rate (for tonic stimulation);
intraburst frequency, or interburst frequency, or the number of pulses in each burst, for burst stimulation.

Various combinations of these features are possible, making the number of possible ways to vary a single therapy program quite large. Still further, neuromodulation system can also store more than one programmed therapy, and may define a plurality of areas for stimulation so that multiple programs can be running at once—that is, referring to FIG. 2, the area at 58 could be stimulated according to a first plan, and the area at 60 could be stimulated according to a second plan, with both the first and second plans running simultaneously or being alternated in any desired pattern. That is, each area may have independently controlled duty cycle and other characteristics.

The plethora of variables is such that testing and optimization of therapy can be enhanced with automated methods and with a variety of tools.

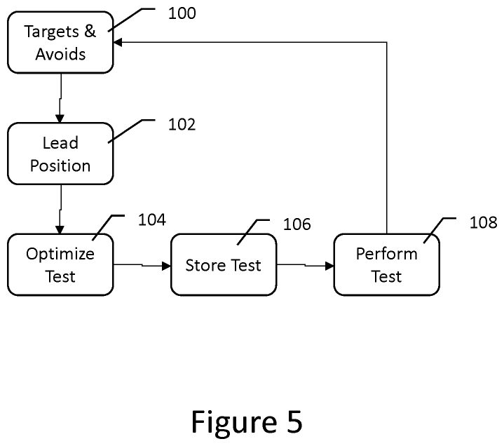

FIG. 5 shows a method of stimulation planning for spatial purposes. Here, structures near the lead, such as, for a DBS lead, structures in the brain, are identified as target structures for therapy, and avoid structures that are not to be stimulated, as indicated at 100. The targets are areas or volumes of tissue that the physician wishes to deliver electrical pulses to. The avoid structures are areas or volumes of tissue that the physician does not want to deliver electrical pulses to include, for example, structures associated with negative outcomes and/or side effects. Target and avoid structures may be initially identified prior to testing using a brain atlas for example, as well as imaging studies of the patient. The lead position 102 is also determined, typically, using imaging studies following implant of the lead. Though not shown above, most implanted neuromodulation leads include one or more radiopaque markers that can be used to determine lead position clearly from an X-ray, as well as lead orientation.

Test parameters can then be optimized. Optimization may include not only consideration of target and avoid structures but also identified therapy parameters that minimize the volume of tissue that is subjected to electrical fields, particularly electrical fields above the estimated activation threshold of tissue. An activation threshold may be understood as a threshold at which an action potential is likely to result from the electrical field being applied for a given duration of time. Longer durations (pulse width) and higher amplitude, for example, will stimulate more tissue (all else being equal) than shorter durations and lower amplitudes. The aim is to avoid unnecessarily causing action potentials, even if no side effect is observed, as a precaution against (potentially unknown) long-term negative effects. In some examples, a grid of the surrounding tissue is defined, referred to as “voxelization” of the tissues. The voxels may further be characterized as residing inside of or outside of target and avoid structures. A histogram that estimates which voxels would be stimulated above the activation threshold are counted, to yield a metric or score for a proposed set of therapy parameters which weighs the desirable stimulation of target structures against undesirable stimulation of avoid structures and overall stimulation (background). Some examples of optimization using spatial considerations are described in U.S. Pat. No. 11,195,609, and U.S. patent application Ser. Nos. 18/804,678 and 18/804,599, both filed Aug. 14, 2024, the disclosures of which are incorporated herein by reference for details of voxelization, histogram, and optimization procedures.

By optimization, with respect to, for example, the identification of a relationship between a therapy parameter and a therapy outcome, benefic, effect, side effect, etc., when a therapy benefit is analyzed, the optimization may seek therapy parameters that yield more of the therapy benefit. For example, therapy that has a post-stimulation benefit can be optimized by selecting parameters which, based on the previous testing, are expected to cause the post-stimulation benefit to persist for a longer period of time than other therapy parameters. If a cost function is used, as discussed further below, optimized therapy parameters that minimize the cost function may be selected by first identifying a relationship between the therapy parameters and the therapy outcome using the data from previous testing (which may also be combined with other data from other patients, such as population based data or data from similarly situated patients), then then selecting the therapy parameters that minimize the cost function.

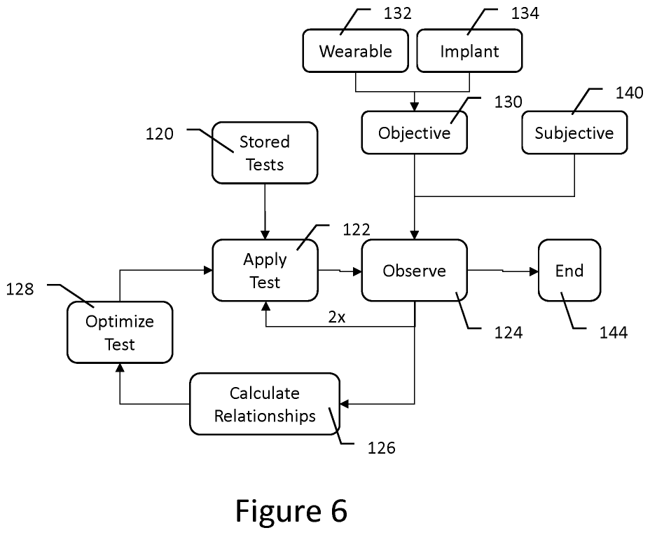

The optimized test parameters are then stored at 106 and tested as indicated at 108. Some examples may include performing the test in-clinic at 108 and reverting to block 100 to update the known target and avoid structures using any observed response, whether beneficial or not, of the patient. Some examples may instead store the test programs to be later used by a patient while out-of-clinic, if desired, such as for use at home. A plurality of test programs may be stored at block 106, as desired. Modern implantable systems are typically capable of storing several therapy regimens (including test programs) for later use and, furthermore, the patient remote controls that are available for use may include general purpose or dedicated devices, such as smart phones, that can store dozens or hundreds of programs, if desired. Thus, a plurality of test programs can be configured and the patient sent home to test the programs on themselves, at the direction of or under advice of their physician. Test results can be determined using inputs from the patient received by a clinician or at a patient remote control, as well as data captured by wearable or implantable devices configured to capture any of electrical signals and/or patient movements or other measurables as described previously. FIG. 6 shows an illustrative method of testing and/or optimizing programs. Stored tests at 120 may be used by the system, such a stored test programs from performing a method as in FIG. 5, for example and without limitation. A selected one of the stored tests is applied at block 122, and patient response is observed at 124. Again, patient response may use patient input data to a clinician, or at a patient remote control, as well as any measurable response using implanted or wearable devices. This sequence can be repeated at least once (thus the “2×” shown between 122 and 124), so that two tests and two patient responses are available.

Relationships between patient response and test parameters are then calculated at 126. If no patient response occurs, the system may return from 124 to 122 to modify test parameters until a response can be observed, or until a stop condition is reached, as desired (a stop condition may include a maximum amplitude for example, or a quantity of tests, as desired). For example, adjustments may include increasing amplitude until a patient response is observed or a maximum amplitude is reached.

With at least two tests having been applied, and two corresponding patient responses obtained, relationships calculated at 126 may include determining what changed from one applied stimulation test to another, and how such changes influence patient response. Relationships can take various forms. The method may be performed, for example and without limitation, by having the implantable pulse generator use the lead to apply the tests at block 122. Observations can be made as indicated using measurable inputs from a wearable device 132 and/or an implanted device 134 (the pulse generator or a separate implantable or ingestible device), and the external programmer (CP or RC) may obtain data from the wearable or implanted or ingestible device. The calculation of relationships at 126 and test optimization 128 may be performed by the external programmer (CP or RC); alternatively, in some illustrative examples, the implantable pulse generator may have a controller for performing such steps. The calculation and optimization may include quantifying the relationship and then using a cost function in some examples.

In an example, the therapy response that is used is a persistence of a therapy effect. For example, some therapies may continue to affect the patient after the electrical stimulation stops. The length of time from when a therapy output stops to when the therapy effect stops may be determined by, for example, observing a sensor output indicative of therapy response, or obtaining a response from the patient such a on a remote control or by a clinician. In a specific example, a patient may receive pain relief form a therapy being delivered, and so a patient may be queried using the patient remote control to determine when analgesic effects of therapy cease to be observed by the patient. In another example, a therapy that causes patient tremor to cease may be observed using a wearable device that senses patient movement, in particular the tremor to be treated, and so an output of the wearable device can be used to measure the time at which the tremor is observed after therapy delivery stops.

Variation of the first and second therapies may take several forms. For example, burst therapy can be issued using an interburst period, an intraburst period, and a number of stimuli in each burst. The therapy parameter that varies can be any of the interburst period, the intraburst period, and/or the number of stimuli in each burst. For any of these burst therapies, the therapy would have a first burst and a last burst, and the time when the therapy output stops is at the end of the last burst. Then, for any of these examples, the persistence can be characterized in a formula:

$$L = f ( p )$$

Where L is the inverse of therapy persistence, and p is the controlled parameter which varies from one test to the next. The goal then is to seek a controlled parameter what achieves the minimized value for L (L being an inverse, this would be the longest persistence), within an applicable set of constraints, such as maximum and minimum values for the controlled parameter. The function, f, may be linear or non-linear. In some examples, the function may be identified from a best-fit analysis. The solution may not take a closed form, and the empirical data gathered during testing on a patient and/or class of patients may be used to determine an empirical solution for f, leading to an empirically determined optimum solution. As a simple example, f (p) may be of the form A+B*p where A and B are best fit parameters. Some examples may use a first order or second order equation to characterize the relationship. A polynomial can be identified, if desired, such as a quadratic formula.

A multi-variable approach can be taken, if desired, find optimized parameters for each of multiple parameters, optionally taking one at a time. That is, in an example, the interburst period that yields a longest persistence can be found first, then an intraburst period that yields a longest persistence, and so forth with any of the desired parameters. Thus, by holding a first (or more) parameter constant while varying a second parameter and then switching the analysis to hold the second parameter constant while varying the first parameter, a larger data set and more degrees of analysis can be established.

Still further examples may select a different measurable therapy response. In another example, the therapy response is a delay to onset of a therapy effect determined as a length of time when a therapy output starts to when the therapy effect starts and/or stabilizes. Stabilizes, as used here, indicates the therapy effect (which is a beneficial effect in some examples, though other examples may use a side effect, optionally) is not only observed but becomes consistent. As an example, cessation of a tremor may be determined using a wearable device that the patient places, for example, on a hand subject to tremor, and onset can be determined when the wearable device determines cessation of the tremor is both observed and has lasted at least a minimum duration (1-5 seconds, for example). This delay may be described as a wash-in period. In the above formula, using the delay to onset of therapy effects, then L would be the inverse of the delay.

The therapy response may be a measure of neural activity detected by the implantable neuromodulation system itself or a separate implantable or wearable system. For example, some therapy may be directed to address parasympathetic nervous system activity. Sensing activity at the vagus nerve may be useful to determine effect of a delivered therapy on the parasympathetic nervous system, and so a measure of such activity may be obtained from the vagus nerve at any suitable location, such as on the anterior neck or anywhere that the vagus nerve can be monitored. Likewise, in a deep brain stimulation system having the at least one lead implanted in the patient's brain, the therapy response can be determined from a measurable electrical activity in the brain of the patient. Sensing any of alpha, beta or other brain waves can be used, as desired. Beta oscillation, for example, may be measured to determine delay to onset of therapy effect, and or persistence of the therapy effect.

In another example, beta waves may be monitored for the degree of effect on beta waves. For example, long duration bursts of Beta wave activity is thought to indicate an undesired outcome. A set of Beta waves may be captured before, during and after a test therapy to identify differences and formulate a ratio of long and short duration Beta waves, thus providing a measure of therapy effectiveness during and after the test therapy, as compared to the ration observed at baseline, before the test stimulation is delivered.

Some examples further implicate spatial variation. For example, a first programmed therapy is output with a first spatial pattern of stimulation, and the second programmed therapy is output with a second spatial pattern of stimulation, and the relationship characterizes a therapy effect as a function of spatial pattern of stimulation. The change from the first spatial pattern to the second spatial pattern may be a change in the central point of stimulation from one position to another, or a change in the spread of the output therapy.

In an example, the central point of stimulation can be tested sequentially by first selecting a particular fractionalization (both spread of the therapy signal and central point of stimulation would thus be determined), and testing with therapy on and then off to determine time from therapy start to therapy effect, and time from cessation of therapy to cessation of therapy effect, for example. Then a new, different fractionalization would be selected.

Stimulation may vary in both space and time (spatio-temporal variation), as for example when using a coordinated reset therapy. For example, coordinated reset therapy is described in U.S. PG Pat. Pub. No. 2018/0345022, the contents of which are incorporated herein by reference.

For spatio-temporal optimization or in general for multi-dimensional optimization, one may not want to optimize sequentially but would rather optimize directly within the high dimensional space. Thus, for example, choice of electrodes or fractionalization, and choice of burst duration can constitute a 2 variable optimization carried out using, for example, and without limitation, gradient descent or other optimization algorithms. A contour map might be useful for depiction of this multidimensional optimization and can be displayed to the user via RC or CP graphical user interface, if desired.

In some examples, the duration of therapy can be the variable. Thus, a first test therapy may be applied for a first period of time, and the second test therapy for a second period of time. The optimization may then select the duration that provides an enhanced persistence.

Further balancing can be performed, such as, for example, seeking to minimize a cost function, X, where:

$$X = {w}_{1} * f ( p ) + {w}_{2} * A$$

Where f(p) is a benefit of the therapy, such as an inverse of persistence, and A is a cost of the therapy, calculated for example by energy expenditure or by a quantification of the duty cycle (duration of therapy on time to total time, for example). Weighting factors w₁ and w₂ can then be used to weight the therapy benefit and cost (a negative coefficient may be a component of weight w₁ and/or f(p) in the above so that the benefit of therapy reduces the value of X, as the cost function will then be minimized). Use of “duty cycle” as a cost of therapy may be based on the understanding that the body, and neural system, of a patient will adjust to therapy (a plastic response), and that therapy can lose efficacy over time as a function of the amount of therapy that is delivered to the patient. That is, the patient's neural system is less likely to accommodate to a given therapy over time if the therapy is delivered at a lower duty cycle, all else being equal. Duty cycling can be used, in some examples, as part of the long term therapy plan for therapy having relatively longer lasting after effects, such as Coordinated Reset therapy.

Side effects may be considered in the optimization. For some more or less binary effects, such as face pulling side effect which may occur with movement disorder therapy, the side effect can provide a search boundary. For other effects, such as those affecting the nervous system at large (elevated heart rate, sweating, reduced heart rate variability, etc.) the cost function X may include additional terms to account for these other side effects, either individually or as a grouping of “costs.” For example:

$$X = {w}_{1} * f ( p ) + {w}_{2} * A + {\sum }_{i = 0}^{n} {c}_{i} * {M}_{i}$$

Where f(p), A, w₁ and w₂ are each as previously described, and the summation allows the cost (cᵢ) for each measure of a side effect (Mᵢ) to be accounted for a set of n side effects. Any suitable number of side effects can be accounted for. If desired, benefits can also be treated as a summation, if more than one distinct effect is defined. These variables can be adjusted by the physician and/or user as desired.

The test is then optimized at 128, to yield a new therapy test parameter set. This newly generated therapy test parameter set may then be applied at 122 with observation at 124.

The observing step 124 may use objective inputs 130, obtained for example, from wearable devices 132 and/or implanted systems 134 (the implant that delivers therapy, or a separate implant can be used at 134). Any of the various wearable or implantable systems described previously may be used at 130 to observe the patient's therapy response. Subjective inputs 140 may also be used, such as by querying the patient using the patient remote control.

The method can end at 144 once a stop condition is reached. A stop condition may be reached in a simple example once at least two stored tests 120 are performed, and at least one optimized test 128 is performed. While this suggests three tests, in an alternative example, a single stored test program is executed, and a predetermined set of relationships are used at 126 to identify an optimized test. The predetermined set of relationships may come from observations made across a patient population, for example. Other stop conditions may include determining whether more than one optimized tests have been calculated and whether the therapy benefit (or other analysis endpoint, such as a minimized cost function) has peaked and is therefore unlikely to further improve.

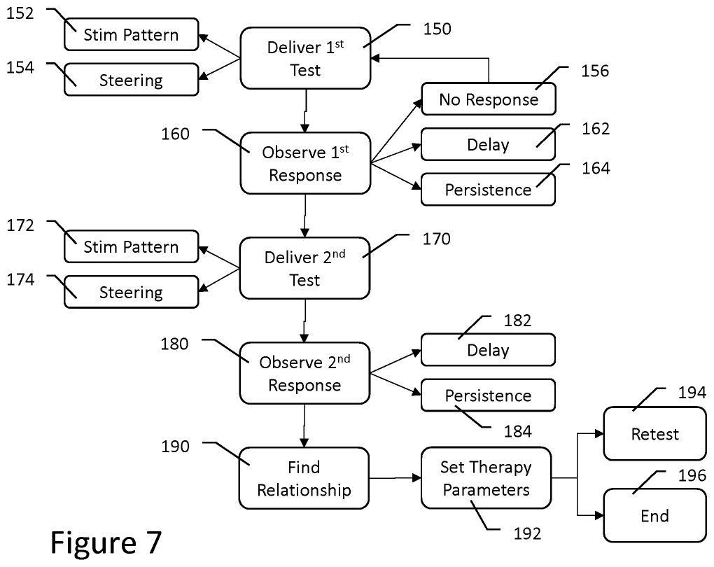

FIG. 7 shows another illustrative method. At block 150, a first test therapy is delivered, such as by using a system as illustrated in FIG. 1 with a lead as in FIG. 2. Block 150 may be used with other system (VNS, SNS, SCS, etc.), and different lead designs, including linear and/or paddle leads as well.

The first test stimulation at 150 may use a particular stimulation pattern 152. The stimulation pattern may be a selection of pulse parameters, such as a particular set of pulse amplitude, pulse width, pulse repetition rate, and quantity of pulses or duration of therapy. The stimulation pattern may be further characterized as a burst pattern, having a selected set of intraburst period, inter burst period, amplitude, pulse width, in-burst pulse quantity, and quantity of bursts parameters. Pulse shape may also be selected, including use of active recharge or passive recharge, and square or non-square wave parameters.

The first test stimulation may have a selected set of steering parameters 154, defining which electrodes are used in the stimulation and what amplitude (current or voltage) and polarity (anode, cathode, off) each electrode has. Steering parameters 154 may be defined at the electrode level or may yield a particular central point of anodic and/or cathodic stimulation and/or spread as previously described.

A first response to the first test stimulation is observed at 160. Optionally, this may include observing effectiveness generally—that is, whether any effect on the patient is observed, and if not, returning to re-deliver the first test stimulation at a higher amplitude, intensity, pulse width or other parameter, if desired, as indicated at 156. If a response is observed at 160, the delay between initiation of the stimulation in block 150 to the observed response may be determined, as indicated at 162. Additionally or instead of block 162, the persistence of the therapy effect can be observed at 164, in which a time from termination of the first test stimulation (which may include multiple pulses, as described above, including a burst therapy) to the end of the observed response is determined.

Block 160 may be performed using any of the sensors and/or inputs described previously, such as internal, implantable, and/or wearable devices and/or sensors, and/or patient inputs obtained by an external device such as the patient remote control or another device.

A second test stimulation is then delivered at block 170. The second test stimulation has a second stimulation pattern 172 and a second steering 174, each characterized using one or more of the various parameters described above for block 152 and 154, respectively.

A second response is observed at 180, this time the response to the second test stimulation from block 170. The test stimulation block 170 may be executed with an increase in therapy amplitude, intensity and/or energy until a response is observed, if desired. When a second response is observed, the delay 182 and/or persistence 184 of the patient's response to the second test stimulation is determined. Block 180 may be performed using any of the sensors and/or inputs described previously, such as internal, implantable, and/or wearable devices and/or sensors, and/or patient inputs obtained by an external device such as the patient remote control or another device.

A relationship between a stimulation parameter which differentiates the first test stimulation from the second test stimulation and the observed response is then identified or characterized at 190. One or more new set(s) of therapy parameter is then set at 192, and subject to retest at 194 (returning, for example, to one of block 150 or 170) with observation of patient response. The cycle may end at 196, with or without the retest 194.

In a specific example, the first test stimulation uses therapy parameters including a selected burst therapy regimen having a first intraburst period, and the second test stimulation uses therapy parameters that are different from the first test stimulation by using a second intraburst period. The first test is observed at 160 to yield a first persistence, and the second test is observed at 180 to yield a second persistence. Supposing, for illustration and not limitation, that the data is as shown here:

| | Intraburst Period | Persistence |
|---|---|---|
| Test 1 | 20 ms | 100 seconds |
| Test 2 | 25 ms | 150 seconds |

Noting that the basic equation to resolve is of the form:

$$L = f ( p )$$

Where L is the inverse of the persistence, and p is the controlled variable, then the solution is as follows:

$$L = A * p + B = - 0 \text{.0666} * p + 0.023$$

Where L is in units of inverse seconds, and p is in units of seconds, and the coefficient A has units of inverse seconds squared and B in inverse seconds. A third test at 30 milliseconds intraburst period would then be recommended, in this example, and would be expected to extend the persistence to over 300 seconds. This numerical example is provided for illustration and is not intended to be limiting. Rather than a linear function, a polynomial function can be used, such as by finding a best fit to a quadratic function. In still other examples empirical minimization may be used, as described previously.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient with an implantable neuromodulation system having a pulse generator and at least one lead having a plurality of electrodes thereon, the method comprising:
delivering a first programmed therapy to the patient with first parameters using the pulse generator and at least one lead;
observing a first response to the delivered first programmed therapy;
delivering a second programmed therapy to the patient with second parameters using the pulse generator and at least one lead, the second parameters having at least one changed parameter relative to the first parameters;
observing a second response to the delivered second programmed therapy;
identifying a relationship between the changed parameter and a therapy response from the first programmed therapy, the first response, the second programmed therapy, and the second response;
calculating an optimized therapy using the identified relationship; and
delivering the optimized therapy to the patient.

2. The method of claim 1, wherein the therapy response is a persistence of a therapy effect determined as a length of time from when a therapy output stops to when the therapy effect stops.

3. The method of claim 2, wherein the first programmed therapy is a burst therapy having an interburst period, an intraburst period, and a number of stimuli in each burst, and the changed parameter is the interburst period.

4. The method of claim 2, wherein the first programmed therapy is a burst therapy having an interburst period, an intraburst period, and a number of stimuli in each burst, and the changed parameter is the intraburst period.

5. The method of claim 2, wherein the first programmed therapy is a burst therapy having an interburst period, an intraburst period, and a number of stimuli in each burst, and the changed parameter is the number of stimuli in each burst.

6. The method of claim 2, wherein the first programmed therapy is a burst therapy comprising a series of bursts including a first burst and a last burst, and the time when the therapy output stops is at the end of the last burst.

7. The method of claim 1, wherein the therapy response is a delay to onset of a therapy effect determined as a length of time when a therapy output starts to when the therapy effect starts.

8. The method of claim 1, wherein the therapy response is determined from a measure of patient movement determined using one or more of a wearable device, or a motion sensor of the implantable neuromodulation system.

9. The method of claim 1, wherein the implantable neuromodulation system is a deep brain stimulation system having the at least one lead implanted in the patient's brain, and the therapy response is determined from a measurable electrical activity in the brain of the patient measured either with the implantable neuromodulation system or by a separate device.

10. The method of claim 1, wherein the first programmed therapy is output with a first spatial pattern of stimulation, and the second programmed therapy is output with a second spatial pattern of stimulation, the changed parameter is the spatial pattern, and the relationship characterizes a therapy effect as a function of spatial pattern of stimulation.

11. The method of claim 10, wherein the first spatial pattern has a first spread of stimulation outputs, the second spatial pattern has a second spread of stimulation outputs, and the relationship characterizes the therapy effect as a function of spread of stimulation outputs.

12. The method of claim 10, wherein the first spatial pattern has a first center of stimulation outputs, and the second spatial pattern as a second center of stimulation outputs, and the relationship characterizes the therapy effect as a function of the center of stimulation outputs.

13. The method of claim 1, wherein the relationship characterizes a persistence of the therapy effect as a function of spatial and temporal parameters of the stimulation outputs.

14. A method of treating a patient with an implantable neuromodulation system having a pulse generator and at least one lead having a plurality of electrodes thereon, the method comprising:
testing a first programmed therapy by:
delivering the first programmed therapy to the patient;
observing a first change in a chosen symptom of the patient;
ceasing to deliver the first programmed therapy;
observing a second change in the chosen symptom of the patient; and
determining a first duration related to the first programmed therapy and the chosen symptom;
testing a second programmed therapy by:
delivering the second programmed therapy to the patient;
observing a third change in the chosen symptom of the patient;
ceasing to deliver the second programmed therapy;
observing a fourth change in the chosen symptom of the patient; and
determining a second duration related to the first programmed therapy and the chosen symptom;
identifying a relationship between a therapy parameter and a therapy result from the first programmed therapy, the first duration, the second programmed therapy, and the second duration;
calculating an optimized therapy using the identified relationship; and
delivering the optimized therapy to the patient.

15. The method of claim 14, wherein the pulse generator is configurable to deliver electrical stimuli using a plurality of spatial patterns, the first programmed therapy is output with a first spatial pattern, and the second programmed therapy is output with a second spatial pattern different from the first spatial pattern, and the relationship characterizes a therapy effect as a function of spatial pattern of stimulation.

16. The method of claim 15, wherein the first spatial pattern has one of:
a first central point of stimulation which is different from a second central point of stimulation of the second spatial pattern; or
a first spread of electrical outputs in the plurality of electrodes which is different form a second spread of electrical outputs in the plurality of electrodes.

17. The method of claim 14, wherein each of the first programmed therapy and the second programmed therapy are burst therapies each having an interburst period, an intraburst period, and a number of stimuli in each burst, and wherein the first programmed therapy uses a first interburst period and the second programmed therapy uses a second interburst period different from the first interburst period.

18. The method of claim 14, wherein each of the first programmed therapy and the second programmed therapy are burst therapies each having an interburst period, an intraburst period, and a number of stimuli in each burst, and wherein the first programmed therapy uses a first intraburst period and the second programmed therapy uses a second intraburst period different from the first intraburst period.

19. The method of claim 14, wherein each of the first programmed therapy and the second programmed therapy are burst therapies each having an interburst period, an intraburst period, and a number of stimuli in each burst, and wherein the first programmed therapy uses a first number of stimuli in each burst and the second programmed therapy uses a second number of stimuli in each burst different from the first number of stimuli in each burst.

20. An implantable neuromodulation system comprising:
an implantable pulse generator;
at least one implantable lead having a plurality of electrodes thereon; and
an external programmer adapted to communicate with and provide therapy instructions to the implantable pulse generator, wherein the system is configured to:
deliver a first programmed therapy to the patient with first parameters using the pulse generator and at least one lead;
observe a first response to the delivered first programmed therapy;
deliver a second programmed therapy to the patient with second parameters using the pulse generator and at least one lead, the second parameters having at least one changed parameter relative to the first parameters;
observe a second response to the delivered second programmed therapy;
identify a relationship between the changed parameter and a therapy response from the first programmed therapy, the first response, the second programmed therapy, and the second response;
calculate an optimized therapy using the identified relationship; and
deliver the optimized therapy to the patient.
